(19) United States
(12) Patent Application Publication
(10) Pub. No.: US 2014/0031713 A1
(43) Pub. Date: Jan. 30, 2014

(54) TISSUE MASS INDICATOR DETERMINATION

(71) Applicants: Richelle Leanne Gaw, Muenchen (DE); Brian William Ziegelaar, Carina Heights (AU)

(72) Inventors: Richelle Leanne Gaw, Muenchen (DE); Brian William Ziegelaar, Carina Heights (AU)

(73) Assignee: Impedimed Limited, Pinkenba, Queensland (AU)

(21) Appl. No.: 13/983,271

(22) PCT Filed: Jan. 31, 2012
(86) PCT No.: PCT/AU2012/000075

§ 371 Date: Oct. 11, 2013

(30) Foreign Application Priority Data
Feb. 3, 2011 (AU) 2011900340

(51) Int. Cl. A61B 5/053; A61B 5/00

(52) U.S. Cl. CPC A61B 5/0537; A61B 5/7225
USPC 600/547

(57) ABSTRACT

A method of determining a measure of lean tissue mass for a segment of a subject, the method including, in a processing system, determining at least one impedance value at at least one frequency, the at least one impedance value representing the impedance of the segment, determining a tissue mass impedance parameter value using the at least one impedance value and determining a tissue mass indicator based at least in part on the tissue mass impedance parameter value.

### BACKGROUND OF THE INVENTION

The present invention relates to a method and apparatus for use in determining a tissue mass indicator indicative of a lean tissue mass, and which in one example can be used to identify changes in tissue mass.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

There is a paucity of research addressing practical and reliable methods to measure changes in lean tissue mass (LTM), which can be important in assessing the health of individuals, as well as monitoring training, such as body building, rehabilitation from injury, or the like. For example, in the elderly lean tissue mass can be used as an indicator of whether the individual is obtaining adequate nutrition, whilst tracking of lean tissue mass can also be useful in assessing the impact of injury, such as paralysis in the limbs of individuals with spinal cord injury (SCI), or the like.

One technique for assessing lean tissue mass involves the use of DEXA (Dual Energy X-ray Absortiometry), which utilises X-ray absorption scanning of a subject to determine attenuation of transmitted X-rays, which in turn allows information regarding the subject's body composition to be determined. In particular, DEXA can be used to determine a subject's bone mineral density, also known as the subject's ash weight. When combined with additional information, such as the subject's weight and intra- and extracellular fluid levels, this can be used to derive a subject's fat mass and fat-free mass.

However, this requires the use of expensive imaging equipment, and subject's the patient to X-ray dosing, which is undesirable.

One existing technique for determining biological parameters relating to a subject, such as fluid levels, involves the use of bioelectrical impedance. This involves measuring the electrical impedance of a subject's body using a series of electrodes placed on the skin surface. Changes in electrical impedance at the body's surface are used to determine parameters, such as changes in fluid levels, associated with the cardiac cycle or oedema, or other conditions which affect body habitus.

WO00/79255 describes a method of detection of oedema by measuring bioelectrical impedance at two different anatomical regions in the same subject at a single low frequency alternating current. The two measurements are analysed to obtain an indication of the presence of tissue oedema by comparing with data obtained from a normal population.

WO2005/122888 describes a method of detecting tissue oedema in a subject. The method includes determining a measured impedance for first and second body segments. An index indicative of a ratio of the extra-cellular to intra-cellular fluid is then calculated for each body segment, with these being used to determine an index ratio based on the index for the first and second body segments. The index ratio can in turn be used to determine the presence, absence or degree of tissue oedema, for example by comparing the index ratio to a reference or previously determined index ratios.

WO2008/138602 describes a method for use in analysing impedance measurements performed on a subject, the method including, in a processing system determining at least one impedance value, representing the impedance of at least a segment of the subject, determining an indicator indicative of a subject parameter using the at least one impedance value and a reference and displaying a representation of the indicator.

### SUMMARY OF THE PRESENT INVENTION

In a first broad form the present invention provides a method of determining a measure of lean tissue mass for a segment of a subject, the method including, in a processing system:
a) determining at least one impedance value at at least one frequency, the at least one impedance value representing the impedance of the segment;
b) determining a tissue mass impedance parameter value using the at least one impedance value; and,
c) determining a tissue mass indicator based at least in part on the tissue mass impedance parameter value.

Typically the tissue mass impedance parameter value is indicative of an intracellular resistance.

Typically the method includes:
a) determining a fluid level impedance parameter value using at least one impedance value; and,
b) determining a fluid level indicator using at least one fluid level impedance value.

Typically the at least one fluid level impedance parameter value is indicative of intracellular and extracellular fluid levels.

Typically the fluid level indicator is determined based on a ratio of intracellular to extracellular resistance.

Typically the method includes determining an intracellular resistance using the formula:

$${R}_{i} = \frac{{R}_{∞} {R}_{0}}{{R}_{0} - {R}_{∞}}$$

Typically the method includes:
a) determining a first impedance parameter value from at least one impedance value measured at a first time;
b) determining a second impedance parameter value from at least one impedance value measured at a second time;
c) in the processing system, determining at least one of the tissue mass indicator and a fluid level indicator using the first and second impedance parameter values.

Typically the method includes, in the processing system, determining the tissue mass indicator using the equation:

Ind=sf(Rᵢ₂−Rᵢ₁)
wherein:
   Ind is the tissue mass indicator
   sf is a scaling factor
   Rᵢ₁ is a first impedance parameter value
   Rᵢ₂ is a second impedance parameter value

Typically the method includes, in the processing system, determining the tissue mass indicator using the equation:

Ind=LTM=C−sfRᵢ
wherein:
   Ind is the tissue mass indicator
   C is a constant
   sf is a scaling factor

Typically the constant C has a value between 10 and 15, and the scaling factor sf has a value of between 0.002 and 0.004.

Typically the constant C has a value of 13.3, and the scaling factor has a value of 0.0033.

Typically at least one of a constant and a scaling factor are determined from a reference population selected based on at least one of:
a) body segment dominance;
b) differences in body segment types;
c) ethnicity;
d) age;
e) gender;
f) weight; and,
g) height.

Typically the method includes determining at least two impedance values including:
a) a first impedance value at a frequency of below 50 Hz; and,
b) a second impedance value at a high frequency of above 100 Hz.

Typically the first impedance value is indicative of the parameter value R₀ and wherein the second impedance value is indicative of the parameter value R_{∞}.

Typically the method includes:
a) determining a plurality of impedance values at respective frequencies; and,
b) using the plurality of impedance values to determine at least one of the tissue mass impedance parameter value and a fluid level impedance parameter value.

Typically the method includes determining impedance parameter values by at least one of:
a) estimating values based on impedance measurements performed at selected respective frequencies;
b) solving simultaneous equations using the plurality of impedance values;
c) extrapolation from a plot of resistance against reactance for the plurality of impedance values;
d) performing a function fitting technique.

Typically the method includes displaying a representation of at least one of the tissue mass indicator and a fluid level indicator.

Typically the method includes in the processing system, causing one or more impedance measurements to be performed.

Typically the method includes, in the processing system:
a) causing at least one drive signal to be applied to the subject;
b) determining at least one signal measured across the subject; and,
c) determining at least one impedance value using an indication of the drive signal and the signal measured across the subject.

Typically the method includes, in the processing system:
a) controlling a signal generator to thereby cause the at least one drive signal to be applied to the subject; and,
b) determining the at least one signal measured across the subject using a sensor.

Typically the method includes:
a) determining a number of impedance measurements, the number of impedance measurements including at least one impedance measurement at each of a number of measurement frequencies; and,
b) determining the impedance parameter value using the number of impedance measurements.

In a second broad form the present invention provides apparatus for use in analysing impedance measurements performed on a subject, the apparatus including a processing system for:
a) determining at least one impedance value at at least one frequency, the at least one to impedance value representing the impedance of a segment of the subject;
b) determining a tissue mass impedance parameter value using the at least one impedance value; and,
c) determining a tissue mass indicator based at least in part on the tissue mass impedance parameter value.

Typically the apparatus includes:
a) a signal generator for applying one or more electrical signals to the subject using a first set of electrodes;
b) a sensor for measuring electrical signals across a second set of electrodes applied to the subject; and,
c) a controller for:
   i) controlling the signal generator; and,
   ii) determining the indication of the measured electrical signals.

Typically the controller includes the processing system.

Typically the processing system includes the controller.

It will be appreciated that the broad forms of the invention can be used in conjunction, and can be used in a wide range of applications, such as monitoring changes in tissue mass during training, rehabilitation and changes in nutrition.

### BRIEF DESCRIPTION OF THE DRAWINGS

An example of the present invention will now be described with reference to the accompanying drawings, in which:—

FIG. 1 is a schematic of an example of impedance determination apparatus;

FIG. 2 is a flowchart of an example of a process for determining a tissue mass indicator;

FIG. 3A is a schematic of an example of a theoretical equivalent circuit for biological tissue;

FIG. 3B is an example of a locus of impedance known as a Wessel plot;

FIG. 3C is a graph of an example of changes in tissue mass indicator over time;

FIG. 3D is a graph of an example of changes in fluid level indicator over time;

FIG. 4 is a flowchart of an example of a process for determining an indicator determining a tissue mass indicator;

FIGS. 5A and 5B are diagrams of examples of electrode positions for use in measuring limb impedances;

FIGS. 5C and 5D are schematic diagrams of examples of electrode positions for use in measuring limb impedances;

FIG. 5E is a schematic diagram of an example of electrode positions for use in measuring a calf impedance;

FIG. 6A to 6C are schematic diagrams of first examples of representations of indicators;

FIG. 7 is a graph of an example of the relationship between the impedance parameter value Rᵢ and lean tissue mass measured using DXA.

### DETAILED DESCRIPTION OF THE PREFERRED EMBODIMENTS

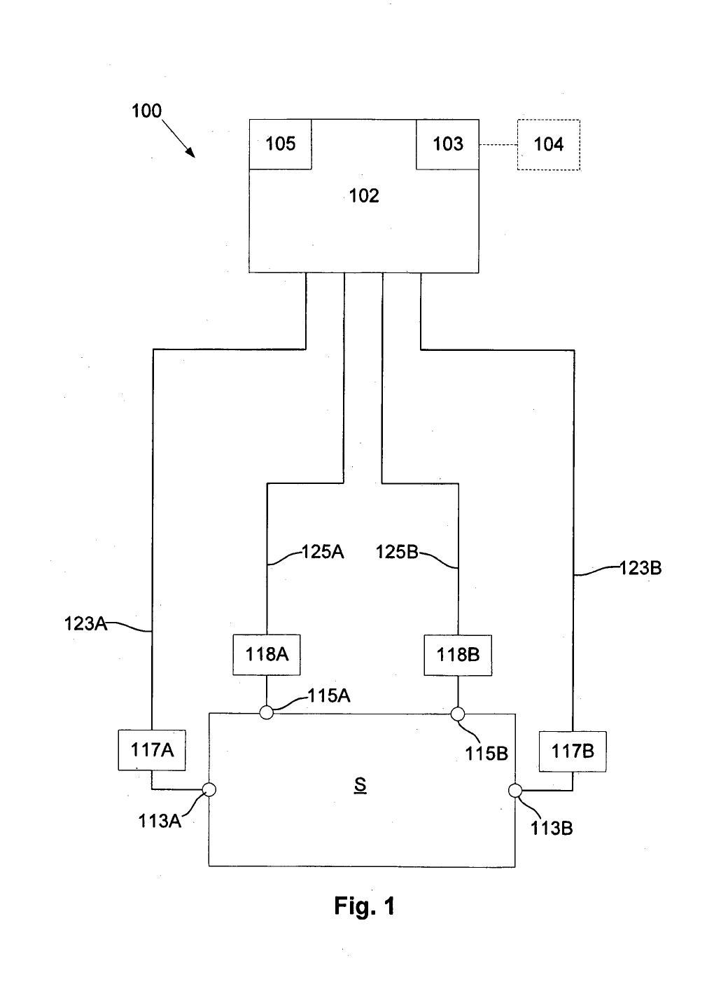

An example of apparatus suitable for performing an analysis of a subject's bioelectric impedance will now be described with reference to FIG. 1.

As shown the apparatus includes a measuring device 100 including a processing system 102, connected to one or more signal generators 117A, 117B, via respective first leads 123A, 123B, and to one or more sensors 118A, 118B, via respective second leads 125A, 125B. The connection may be via a switching device, such as a multiplexer, although this is not essential.

In use, the signal generators 117A, 117B are coupled to two first electrodes 113A, 113B, which therefore act as drive electrodes to allow signals to be applied to the subject S, whilst the one or more sensors 118A, 118B are coupled to the second electrodes 115A, 115B, which act as sense electrodes, allowing signals across the subject S to be sensed.

The signal generators 117A, 117B and the sensors 118A, 118B may be provided at any position between the processing system 102 and the electrodes 113A, 113B, 115A, 115B, and may be integrated into the measuring device 100. However, in one example, the signal generators 117A, 117B and the sensors 118A, 118B are integrated into an electrode system, or another unit provided near the subject S, with the leads 123A, 123B, 125A, 125B connecting the signal generators 117A, 117B and the sensors 118A, 118B to the processing system 102.

It will be appreciated that the above described system is a two channel device, used to perform a classical four-terminal impedance measurement, with each channel being designated by the suffixes A, B respectively. The use of a two channel device is for the purpose of example only, and multiple channel devices can alternatively be used to allow multiple body segments to be measured without requiring reattachment of electrodes. An example of such a device is described in copending patent application number WO2009059351.

An optional external interface 103 can be used to couple the measuring device 100, via wired, wireless or network connections, to one or more peripheral devices 104, such as an external database or computer system, barcode scanner, or the like. The processing system 102 will also typically include an I/O device 105, which may be of any suitable form such as a touch screen, a keypad and display, or the like.

In use, the processing system 102 is adapted to generate control signals, which cause the signal generators 117A, 117B to generate one or more alternating signals, such as voltage or current signals of an appropriate waveform, which can be applied to a subject 5, via the first electrodes 113A, 113B. The sensors 118A, 118B then determine the voltage across or current through the subject 5, using the second electrodes 115A, 115B and transfer appropriate signals to the processing system 102.

Accordingly, it will be appreciated that the processing system 102 may be any form of processing system which is suitable for generating appropriate control signals and at least partially interpreting the measured signals to thereby determine the subject's bioelectrical impedance, and optionally determine other information such as relative fluid levels, or the presence, absence or degree of conditions, such as oedema, lymphoedema, measures of body composition, cardiac function, or the like.

The processing system 102 may therefore be a suitably programmed computer system, such as a laptop, desktop, PDA, smart phone or the like. Alternatively the processing system 102 may be formed from specialised hardware, such as an FPGA (field programmable gate array), or a combination of a programmed computer system and specialised hardware, or the like.

In use, the first electrodes 113A, 113B are positioned on the subject to allow one or more signals to be injected into the subject S. The location of the first electrodes will depend on the segment of the subject S under study. Thus, for example, the first electrodes 113A, 113B can be placed on the thoracic and neck region of the subject S to allow the impedance of the chest cavity to be determined. Alternatively, positioning electrodes on the wrist and ankles of a subject allows the impedance of limbs, torso and/or the entire body to be determined.

Once the electrodes are positioned, one or more alternating signals are applied to the subject S, via the first leads 123A, 123B and the first electrodes 113A, 113B. The nature of the alternating signal will vary depending on the nature of the measuring device and the subsequent analysis being performed.

For example, the system can use Bioimpedance Analysis (BIA) in which a single low frequency signal is injected into the subject S, with the measured impedance being used directly in the determination of biological parameters. In one example, the applied signal has a relatively low frequency, such as below 100 kHz, more typically below 50 kHz and more preferably below 10 kHz. In this instance, such low frequency signals can be used as an estimate of the impedance at zero applied frequency, commonly referred to as the impedance parameter value R₀, which is in turn indicative of extracellular fluid levels.

Alternatively, the applied signal can have a relatively high frequency, such as above 200 kHz, and more typically above 500 kHz, or 1000 kHz. In this instance, such high frequency signals can be used as an estimate of the impedance at infinite applied frequency, commonly referred to as the impedance parameter value R_{∞}, which is in turn indicative of a combination of the extracellular and intracellular fluid levels, as will be described in more detail below.

Alternatively and/or additionally, the system can use Bioimpedance Spectroscopy (BIS) in which impedance measurements are performed at each of a number of frequencies ranging from very low frequencies (4 kHz) to higher frequencies (1000 kHz), and can use as many as 256 or more different frequencies within this range. Such measurements can be performed by applying a signal which is a superposition of plurality of frequencies simultaneously, or a number of alternating signals at different frequencies sequentially, depending on the preferred implementation. The frequency or frequency range of the applied signals may also depend on the analysis being performed.

When impedance measurements are made at multiple frequencies, these can be used to derive one or more impedance parameter values, such as values of R₀, Z_{c}, R_{∞}, which correspond to the impedance at zero, characteristic and infinite frequencies. These can in turn be used to determine information regarding both intracellular and extracellular fluid levels, as will be described in more detail below.

A further alternative is for the system to use Multiple Frequency Bioimpedance Analysis (MFBIA) in which multiple signals, each having a respective frequency are injected into the subject S, with the measured impedances being used in the assessment of fluid levels. In one example, four frequencies can be used, with the resulting impedance measurements at each frequency being used to derive impedance parameter values, for example by fitting the measured impedance values to a Cole model, as will be described in more detail below. Alternatively, the impedance measurements at each frequency may be used individually or in combination.

Thus, the measuring device 100 may either apply an alternating signal at a single frequency, at a plurality of frequencies simultaneously, or a number of alternating signals at different frequencies sequentially, depending on the preferred implementation. The frequency or frequency range of the applied signals may also depend on the analysis being performed.

In one example, the applied signal is generated by a voltage generator, which applies an alternating voltage to the subject S, although alternatively current signals may be applied. In one example, the voltage source is typically symmetrically arranged, with each of the signal generators 117A, 117B being independently controllable, to allow the signal voltage across the subject to be varied.

A voltage difference and/or current is measured between the second electrodes 115A, 115B. In one example, the voltage is measured differentially, meaning that each sensor 118A, 118B is used to measure the voltage at each second electrode 115A, 115B and therefore need only measure half of the voltage as compared to a single ended system.

The acquired signal and the measured signal will be a superposition of voltages generated by the human body, such as the ECG (electrocardiogram), voltages generated by the applied signal, and other signals caused by environmental electromagnetic interference. Accordingly, filtering or other suitable analysis may be employed to remove unwanted components.

The acquired signal is typically demodulated to obtain the impedance of the system at the applied frequencies. One suitable method for demodulation of superposed frequencies is to use a Fast Fourier Transform (FFT) algorithm to transform the time domain data to the frequency domain. This is typically used when the applied current signal is a superposition of applied frequencies. Another technique not requiring windowing of the measured signal is a sliding window FFT.

In the event that the applied current signals are formed from a sweep of different frequencies, then it is more typical to use a signal processing technique such as multiplying the measured signal with a reference sine wave and cosine wave derived from the signal generator, or with measured sine and cosine waves, and integrating over a whole number of cycles. This process, known variously as quadrature demodulation or synchronous detection, rejects all uncorrelated or asynchronous signals and significantly reduces random noise.

Other suitable digital and analogue demodulation techniques will be known to persons skilled in the field.

In the case of BIS, impedance or admittance measurements are determined from the signals at each frequency by comparing the recorded voltage and the current through the subject. The demodulation algorithm can then produce amplitude and phase signals at each frequency, allowing an impedance value at each frequency to be determined.

As part of the above described process, the distance between the second electrodes 115A, 115B may be measured and recorded. Similarly, other parameters relating to the subject may be recorded, such as the height, weight, age, sex, health status, any interventions and the date and time on which they occurred. Other information, such as current medication, may also be recorded. This can then be used in performing further analysis of the impedance measurements, so as to allow determination of the presence, absence or degree of oedema, to assess body composition, or the like.

The accuracy of the measurement of impedance can be subject to a number of external factors. These can include, for example, the effect of capacitive coupling between the subject and the surrounding environment, the leads and the subject, the electrodes, or the like, which will vary based on factors such as lead construction, lead configuration, subject position, or the like. Additionally, there are typically variations in the impedance of the electrical connection between the electrode surface and the skin (known as the “electrode impedance”), which can depend on factors such as skin moisture levels, melatonin levels, or the like. A further source of error is the presence of inductive coupling between different electrical conductors within the leads, or between the leads themselves.

Such external factors can lead to inaccuracies in the measurement process and subsequent analysis and accordingly, it is desirable to be able to reduce the impact of external factors on the measurement process.

One form of inaccuracy that can arise is caused by the voltages across the subject being unsymmetrical, a situation referred to as an “imbalance”. Such a situation results in a significant signal voltage at the subject's body centre, which in turn results in stray currents arising from parasitic capacitances between the subject's torso and the support surface on which the subject is provided.

The presence of an imbalance, where the voltage across the subject is not symmetrical with respect to the effective centre of the subject, leads to a “common mode” signal, which is effectively a measure of the signal at the subject S that is unrelated to the subject's impedance.

To help reduce this effect, it is therefore desirable for signals to be applied to the subject S that they result in a symmetrical voltage about the subject's body centre. As a result, a reference voltage within the subject S, which is equal to a reference voltage of the measurement apparatus, will be close to the effective body centre of the subject, as considered relative to the electrode placement. As the measuring device reference voltage is typically ground, this results in the body centre of the subject S being as close to ground as possible, which minimises the overall signal magnitude across the subject's torso, thereby minimising stray currents.

In one example, a symmetrical voltage about the sensing electrodes can be achieved by using a symmetrical voltage source, such as a differential bidirectional voltage drive scheme, which applies a symmetrical voltage to each of the drive electrodes 113A, 113B. However, this is not always effective if the contact impedances for the two drive electrodes 113A, 113B are unmatched, or if the impedance of the subject S varies along the length of the subject S, which is typical in a practical environment.

In one example, the apparatus overcomes this by adjusting the differential voltage drive signals applied to each of the drive electrodes 113A, 113B, to compensate for the different electrode impedances, and thereby restore the desired symmetry of the voltages across the subject S. This process is referred to herein as balancing and in one example, helps reduce the magnitude of the common mode signal, and hence reduce current losses caused by parasitic capacitances associated with the subject.

The degree of imbalance, and hence the amount of balancing required, can be determined by monitoring the signals at the sense electrodes 115A, 115B, and then using these signals to control the signal applied to the subject via the drive electrodes 113A, 113B. In particular, the degree of imbalance can be calculated by determining an additive voltage from the voltages detected at the sense electrodes 115A, 115B.

In one example process, the voltages sensed at each of the sense electrodes 115A, 115B are used to calculate a first voltage, which is achieved by combining or adding the measured voltages. Thus, the first voltage can be an additive voltage (commonly referred to as a common mode voltage or signal) which can be determined using a differential amplifier.

In this regard, a differential amplifier is typically used to combine two sensed voltage signals Vₐ, V_{b}, to determine a second voltage, which in one example is a voltage differential Vₐ−V_{b} across the points of interest on the subject S. The voltage differential is used in conjunction with a measurement of the current flow through the subject to derive impedance values. However, differential amplifiers typically also provide a “common mode” signal (Vₐ+V_{b})/2, which is a measure of the common mode signal.

Whilst differential amplifiers include a common mode rejection capability, this is generally of only finite effect and typically reduces in effectiveness at higher frequencies, so a large common mode signal will produce an error signal superimposed on the differential signal.

The error caused by common mode signals can be minimised by calibration of each sensing channel. In the ideal case where both inputs of a differential amplifier are perfectly matched in gain and phase characteristics and behave linearly with signal amplitude, the common mode error will be zero. In one example, the two sensing channels of the differential amplifier are digitised before differential processing. It is therefore straightforward to apply calibration factors independently to each channel to allow the characteristics to be matched to a high degree of accuracy, thereby achieving a low common mode error.

Accordingly, by determining the common mode signal, the applied voltage signals can be adjusted, for example by adjusting the relative magnitude and/or phase of the applied signals, to thereby minimise the common mode signal and substantially eliminate any imbalance. An example of this process is described in more detail in copending patent application number WO2009059351.

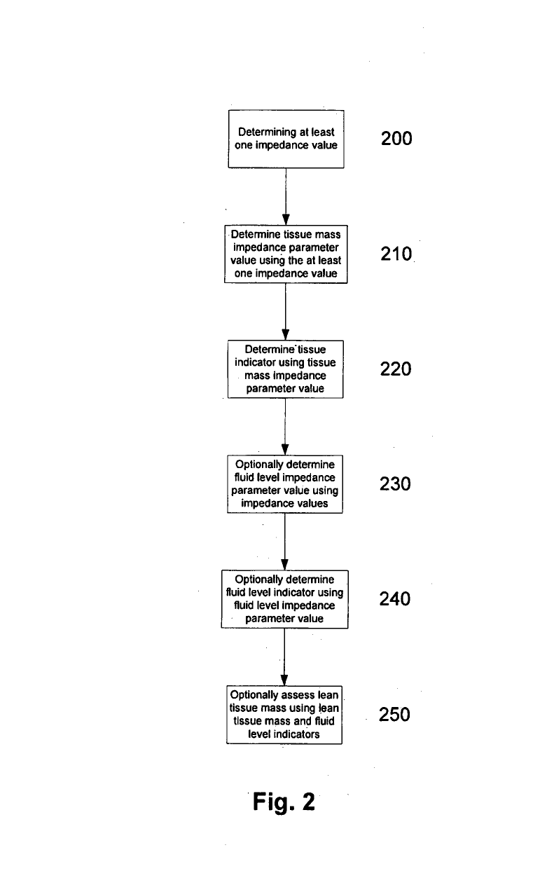

An example of the operation of the apparatus in analysing impedance measurements will now be described with reference to FIG. 2.

In one example, the processing system 102 causes a current signal to be applied to the subject S, with the induced voltage across the subject S being measured, with signals representing the measured voltage and the applied current being returned to the processing system 102 for analysis.

When the process is being used to determine a tissue mass indicator, this is typically performed for at least a segment of the subject S that is suspected of being susceptible to tissue mass loss. This can include any large muscle group, such as the subject's back, legs, thigh, calf or the like.

It will be appreciated that the application of the current and voltage signals may be controlled by a separate processing system that is used in performing the analysis to derive an indicator, and that the use of a single processing system is for the purpose of example only.

At step 200, measured voltage and current signals are used by the processing system 102 to determine at least one impedance value at at least one frequency, the at least one impedance value representing the impedance of a segment of the subject.

At step 210, the at least one impedance value is used by the processing system 102, to determine a tissue mass impedance parameter value. The nature of the tissue mass impedance parameter value can vary, but in general this represents an intracellular impedance Rᵢ, which depends on the amount of intracellular fluid, and hence the amount of lean tissue present.

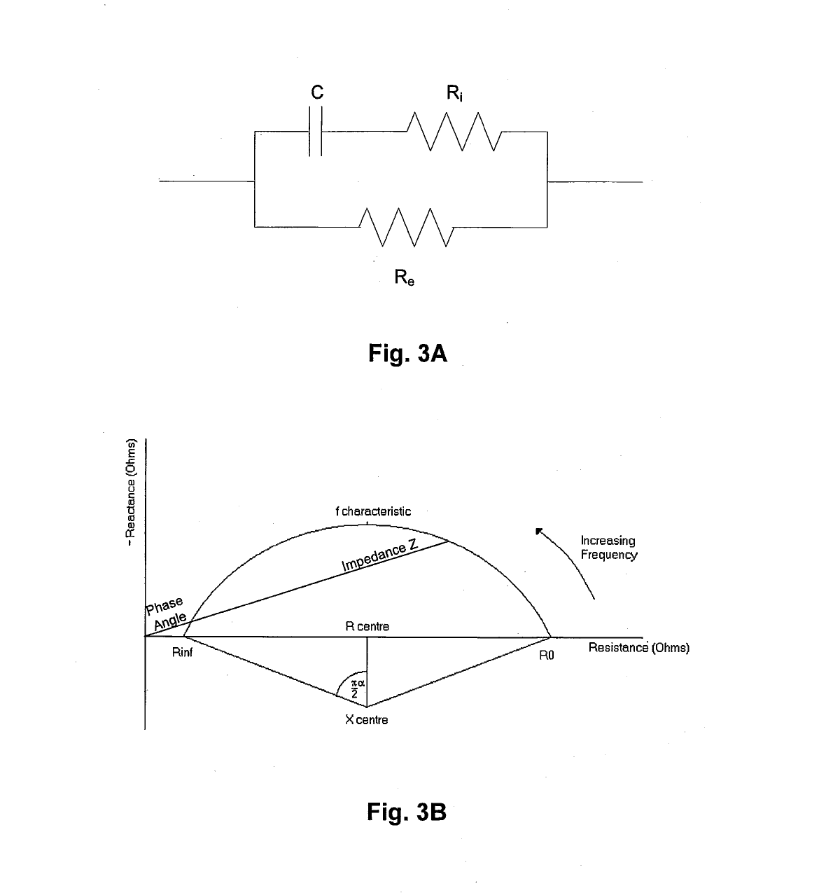

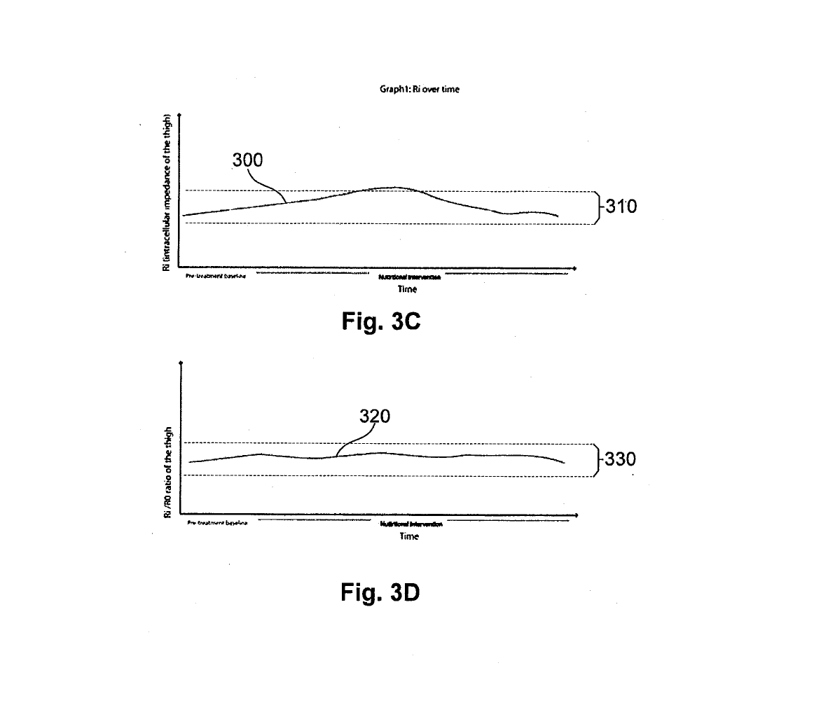

In this regard, FIG. 3A is an example of an equivalent circuit that effectively models the electrical behaviour of biological tissue. The equivalent circuit has two branches that represent current flow through extracellular fluid and intracellular fluid, respectively. The extracellular fluid component of biological impedance is represented by an extracellular resistance Rₑ, whilst the intracellular fluid component is represented by an intracellular resistance Rᵢ and a capacitance C representative of the cell membranes.

The relative magnitudes of the extracellular and intracellular components of impedance of an alternating current (AC) are frequency dependent. At zero frequency the capacitor acts as a perfect insulator and all current flows through the extracellular fluid, hence the resistance at zero frequency, R₀, equals the extracellular resistance Rₑ. At infinite frequency the capacitor acts as a perfect conductor and the current passes through the parallel resistive combination. The resistance at infinite frequency R_{∞} is given by:

$${R}_{∞} = \frac{{R}_{e} {R}_{i}}{{R}_{e} + {R}_{i}} \qquad ( 1 )$$

Hence the intracellular resistance is given by:

$${R}_{i} = \frac{{R}_{∞} {R}_{e}}{{R}_{e} - {R}_{∞}} \qquad ( 2 )$$

Accordingly, the impedance of the equivalent circuit of FIG. 3A at an angular frequency ω, where ω=2π*frequency, is given by:

$$Z = {R}_{∞} + \frac{{R}_{0} - {R}_{∞}}{1 + ( j ω τ )} \qquad ( 3 )$$

where:
   R_{∞}=impedance at infinite applied frequency
   R₀=impedance at zero applied frequency=Rₑ and,
   ρ is the time constant of the capacitive circuit.

However, the above represents an idealised situation which does not take into account the fact that the cell membrane is an imperfect capacitor. Taking this into account leads to a modified model in which:

$$Z = {R}_{∞} + \frac{{R}_{0} - {R}_{∞}}{1 + {( j ω τ )}^{α}} \qquad ( 4 )$$

where: α has a value between 0 and 1 and can be thought of as an indicator of the deviation of a real system from the ideal model.

An example of the typical multi-frequency impedance response is shown in FIG. 3B. As frequency increases, the reactance increases to a peak at the characteristic frequency and then decreases while the resistance continually decreases. This results in a circular locus with the centre of the circle below the x axis, as shown.

The values of impedance parameters X_{c}, R₀, R_{∞}, Z_{c} or α may be determined in any one of a number of manners such as by:
estimating values based on impedance measurements performed at selected respective frequencies;
solving simultaneous equations based on the impedance values determined at different frequencies;
using iterative mathematical techniques;
extrapolation from a plot of resistance against reactance for impedance measurements at a plurality of frequencies (a “Wessel plot” similar to that shown in FIG. 3B);
performing a function fitting technique, such as the use of a polynomial function.

For example, the Wessel plot is often used in BIS devices, which perform multiple measurements over a range of frequencies, such as from 4 kHz to 1000 kHz, using 256 or more different frequencies within this range. A regression procedure is then used to fit the measured data to the theoretical semi-circular locus, allowing values for X_{c}, R₀, R_{∞}, Z_{c} or α to be calculated.

Such a regression analysis is computationally expensive, typically requiring a larger or more expensive device. The regression analysis and also requires a large number of data points, which can cause the measurement process to take a significant amount of time.

Alternatively, a circle fitting technique can be used in which only three measurement points are required. In this technique, three simultaneous equations representing the geometric relationships between points on a circle are solved to allow calculation of the radius (r) and the co-ordinates of the centre of the circle (i, j) as the three parameters which define the circle. From these circle parameters, X_{c}, R₀, R_{∞}, Z_{c} or α are readily computed from geometric first principles.

This circle technique allows a value for X_{c}, R₀, R_{∞}, Z_{c} or α to be derived in a computationally less expensive manner than if a regression analysis is performed, and requires a reduced number of data points allowing a more rapid measurement process.

One potential disadvantage of the use of simultaneous equations is that if one of the impedance measurements is inaccurate for any reason, this can lead to large deviations in the calculated values of X_{c}, R₀, R_{∞}, Z_{c} or α. Accordingly, in one example, impedance measurements are performed at more than three frequencies, with circle parameters for all possible combinations of impedance measurements at three frequencies being calculated. The average can be provided along with the standard deviation as a measure of the goodness of fit of the data to the Cole model. In the event that one of the measurements is inaccurate, this can be accounted for by excluding one or more outlier measurements, such as measurements that deviates the greatest amount from the mean, or measurements differing by more than a set number of standard deviations from the mean, allowing the mean to be recalculated, thereby providing more accurate values.

Whilst this process uses additional measurements, such as four or five measurements, this is still significantly less than the 256 or more frequencies typically performed using a BIS measurement protocol, allowing the measurement process to be performed more quickly.

In one example, the frequencies used are in the range 0 kHz to 1000 kHz, and in one specific example, four measurements are recorded at frequencies of 25 kHz, 50 kHz, 100 kHz, and 200 kHz, although any suitable measurement frequencies can be used.

A further alternative for determining impedance parameter values such as X_{c}, R₀, R_{∞}, Z_{c} or α is to perform impedance measurements at a single frequency, and use these as an estimate of the parameter values. In this instance, measurements performed at a single low frequency (typically less than 50 kHz) can be used to estimate R₀, measurements at a single high frequency (typically more than 100 kHz) can be used to estimate R_{∞}, allowing a value of Rᵢ to be determined using equation (2) above.

The above described equivalent circuit models the resistivity as a constant value and does not therefore accurately reflect the impedance response of a subject, and in particular does not accurately model the change in orientation of the erythrocytes in the subject's blood stream, or other relaxation effects. To more successfully model the electrical conductivity of the human body, an improved CPE based model may alternatively be used.

In any event, it will be appreciated that any suitable technique for determination of the parameter values such as R₀, Z_{c}, R_{∞}, and X_{c} may be used, hence allowing Rᵢ to be derived.

At step 220, the tissue mass impedance parameter value can be used to determine a tissue mass indicator. In one example, the tissue mass indicator is in the form of a numerical value which can be used to determine a relative level of lean tissue mass, and accordingly, in one form, the tissue mass indicator is simply the numerical value of the intracellular resistance Rᵢ.

In another example, a single reading can be scaled, for example based on a scaling factor determined from a study of a reference population. In this regard, by measuring the lean tissue mass of a number of subjects using an alternative technique such as DXA, and then comparing this to the intracellular resistance Rᵢ for the same subjects. A regression analysis can then be performed to determine a relationship between the LTM and Rᵢ. In one example, the regression analysis provides a relationship of the form;

Ind=LTM=C−sfRᵢ (5)
wherein:
   Ind is the tissue mass indicator
   C is a constant
   sf is a scaling factor

In another example, the method includes determining first and second tissue mass impedance parameter values and then determining the tissue mass indicator using the first and second respective tissue mass impedance parameter values. The first and second tissue mass impedance parameter values are typically determined for the same body segment, but at different times, thereby allowing a longitudinal analysis to be performed. It will be appreciated that this is particularly useful in monitoring changes in the tissue mass over time, which can be used to determine the degree of tissue mass loss.

In this example, it will be appreciated that a difference between the first and second tissue mass impedance parameter values can be determined, and scaled by the scaling factor to allow a degree of tissue mass loss to be represented.

Ind=sf(Rᵢ₂−Rᵢ₁) (6)
wherein: Rᵢ₁ is the first tissue mass impedance parameter value
   Rᵢ₂ is the second tissue mass impedance parameter value

Thus, the scaling factor sf can be selected so that the value of the indicator is indicative of the change in LTM.

It will be appreciated that the values for the scaling factor and constant may vary between different populations and body segments. To take this into account, the constant and scaling factor will typically be selected for the subject based on a reference population that is selected to take into account variations in impedance measurements that can arise due to variety of factors such as:
body segment dominance;
differences in body segment types;
ethnicity;
age;
gender;
weight; and,
height.

A study, described in more detail below lead to values of C between 10 and 15, and values of sf of between 0.002 and 0.004. In one particular example C has a value of 13.3, and the scaling factor has a value of 0.0033. However, it will be appreciated that these are for the purpose of example only and are not intended to be limiting.

Changes in the intracellular resistance, and hence changes in the tissue mass indicator, may also depend on other factors in addition to the amount of lean tissue mass, such as changes in global fluid levels within the subject, the presence of oedema, or the like. Accordingly, to take these other factors into account, a second indicator can also be determined for example to reflect changes in fluid levels. In this example, at step 230, one or more impedance values can optionally be used by the processing system 102, to determine one or more fluid level impedance parameter values. The nature of the fluid level impedance parameter value can vary, but in general this is at least partially indicative of the extracellular fluid levels, and more typically both the extracellular and intracellular fluid level. Accordingly, the fluid level impedance parameter values may be based on the impedance R₀ at a zero frequency f₀, although alternatively α may be used, as well as on the intracellular resistance Rᵢ.

At step 240, the fluid level impedance parameter value(s) can be used to determine a fluid level indicator. In one example, the fluid level indicator is in the form of a numerical value that can be used to determine the presence, absence or degree of a condition, such as oedema or lymphoedema, and which is typically based on the ratio of intracellular to extracellular fluid levels. In this instance, the ratio IR is given by:

$$IR = \frac{{R}_{i}}{{R}_{0}} \qquad ( 7 )$$

In one example, the fluid level indicator is given by the numerical value of the ratio IR. However, alternatively, the ratio can be compared to reference values determined from a reference population of healthy individuals, and in particular to the mean ratio value for the reference population. This can be used to determine deviation of the subject's ratio from that of healthy individuals in the reference population, which is in turn indicative of the presence, absence or degree of oedema.

Accordingly, in one example, the fluid level indicator Indf can be determined using the equation (8):

$$Indf = \frac{sf \times ( IR - μ )}{3 σ - μ} \qquad ( 8 )$$

where:
   Indf is the fluid level indicator
   IR is the ratio
   μ is a mean ratio for a reference population
   3σ is three standard deviations for the reference population
   sf is a scaling factor

Typically the scaling factor is selected so that a threshold value indicative of at least one of a presence or absence of oedema is an integer value. Thus, a value such as “10” for the tissue indicator can be used to indicate the presence, absence or degree of oedema. Again, the reference normal population is typically selected to take into account variations in impedance measurements that can arise due to variety of factors such as:
body segment dominance;
differences in body segment types;
ethnicity;
age;
gender;
weight; and,
height.

Alternatively, and/or additionally, changes in the ratio as determined from first and second fluid level impedance parameter values can be determined, and scaled by a scaling factor so that the indicator and the threshold can be a memorable value, such as an integer value, or the like. This can be achieved for example by calculating the fluid level indicators as follows:

Indf=sf(IR₁−IR₂) (9)
where: IR₁ is a first ratio
   IR₂ is a second ratio

By measuring the first and second ratios at different times, this allows variations in the fluid level indicator over time to be monitored. In this instance, the fluid indicator can be compared to a range derived from a normal population to determine if changes in the fluid levels within the subject are indicative of oedema, or are otherwise outside of an expected range.

At step 250, the tissue mass indicator, and optionally the fluid level indicator can be used to assess the lean tissue mass, and in particular, to determine if any changes in the tissue mass indicator are caused by changes in fluid levels in general, or are due to changes in the lean tissue mass.

In this regard, FIGS. 3C and 3D show an example of variations in the tissue mass indicator 300 and fluid level indicator 320, over time. In particular, in this example, the indicators are represented by absolute values of Rᵢ and the ratio Rᵢ/R₀, with a normal expected range of values derived from a study of healthy individuals being shown at 310 and 330, respectively.

In this example, initial (first) readings are established to provide a baseline, with subsequent (second) readings being used to allow changes from this baseline to be monitored. In this example, this shows that the value of Rᵢ, and hence the tissue mass indicator 300, initially rises over time. However, the fluid level indicator 320 stays approximately level, and within the expected range for a normal population 330, thereby indicating that changes in the tissue mass indicator 300 are associated with a change in tissue mass, as opposed to more general changes in fluid levels, the presence of oedema, or the like. Accordingly, in this example, the rise in value of Rᵢ, and hence tissue mass indicator 300, indicates a reduction in lean tissue mass.

A reduction in lean tissue mass can be caused by a range of factors, such as malnutrition, a lack of exercise, or the like, depending on the scenario. Accordingly, in this example, treatment is administered, leading to a subsequent reduction in the value of Rᵢ, and hence the lean tissue mass indicator 300. Again, the fluid level indicator 320 remains substantially unchanged, and within the normal range 330, thereby indicating that the reduction in tissue mass indicator 300 is associated with an increase in tissue mass, and hence that treatment is successful.

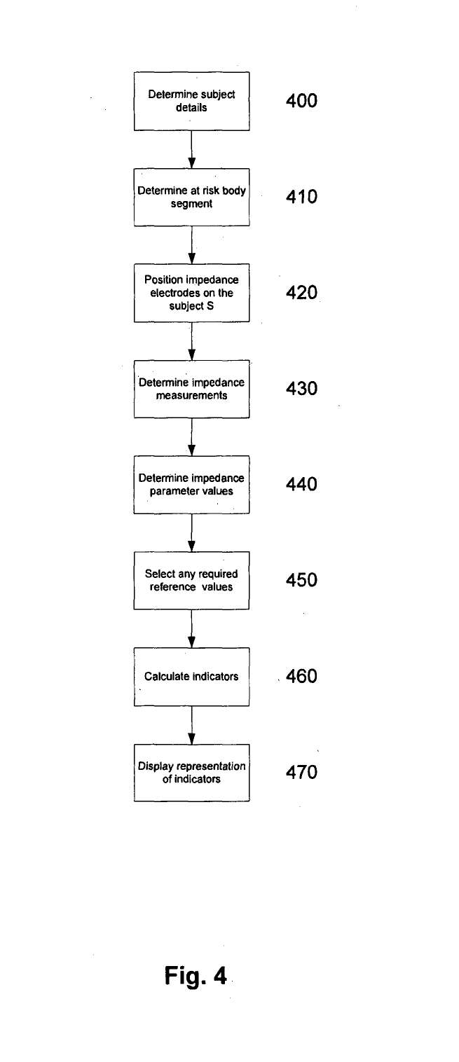

An example of the process for performing impedance measurements to determine a tissue mass indicator for assessing tissue mass changes, and optionally a fluid level indicator for assessing fluid levels, will now be described in more detail with reference to FIG. 4.

In this example, at step 400 subject details are determined and provided to the processing system 102. The subject details will typically include information such as limb dominance, details of any medical interventions, as well as information regarding the subject such as the subject's age, weight, height, sex, ethnicity or the like. The subject details can be used in selecting a suitable reference normal population, as well as for generating reports, as will be described in more detail below.

It will be appreciated that the subject details may be supplied to the processing system 102 via appropriate input means, such as the I/O device 105. Thus, each time a subject measurement is performed this information can be input into the measuring device 100.

However, more typically the information is input a single time and stored in an appropriate database, or the like, which may be connected as a peripheral device 104 via the external interface 103. The database can include subject data representing the subject details, together with information regarding previous tissue mass indicators, baseline impedance measurements recorded for the subject, or the like.

In this instance, when the operator is required to provide subject details, the operator can use the processing system 102 to select a search database option allowing the subject details to be retrieved. This is typically performed on the basis of a subject identifier, such as a unique number assigned to the individual upon admission to a medical institution, or may alternatively be performed on the basis of name or the like. Such a database is generally in the form of an HL7 compliant remote database, although any suitable database may be used.

In one example, the subject can be provided with a wristband or other device, which includes coded data indicative of the subject identifier. In this case, the measuring device 100 can be coupled to a peripheral device 104, such as a barcode or RFID (Radio Frequency Identification) reader allowing the subject identifier to be detected and provided to the processing system 102, which in turn allows the subject details to be retrieved from the database. The processing system 102 can then display an indication of the subject details retrieved from the database, allowing the operator to review these and confirm their accuracy before proceeding further.

At step 410 one or more body segments of interest are determined. This may be achieved in any one of a number of ways depending on the preferred implementation. Thus, for example, the affected limb can be indicated through the use of appropriate input means, such as the I/O device 105. Alternatively this information can be derived directly from the subject details, which may include an indication of an at risk body segment, or details of any medical interventions performed or injuries incurred, which are in turn indicative of the at risk body segment.

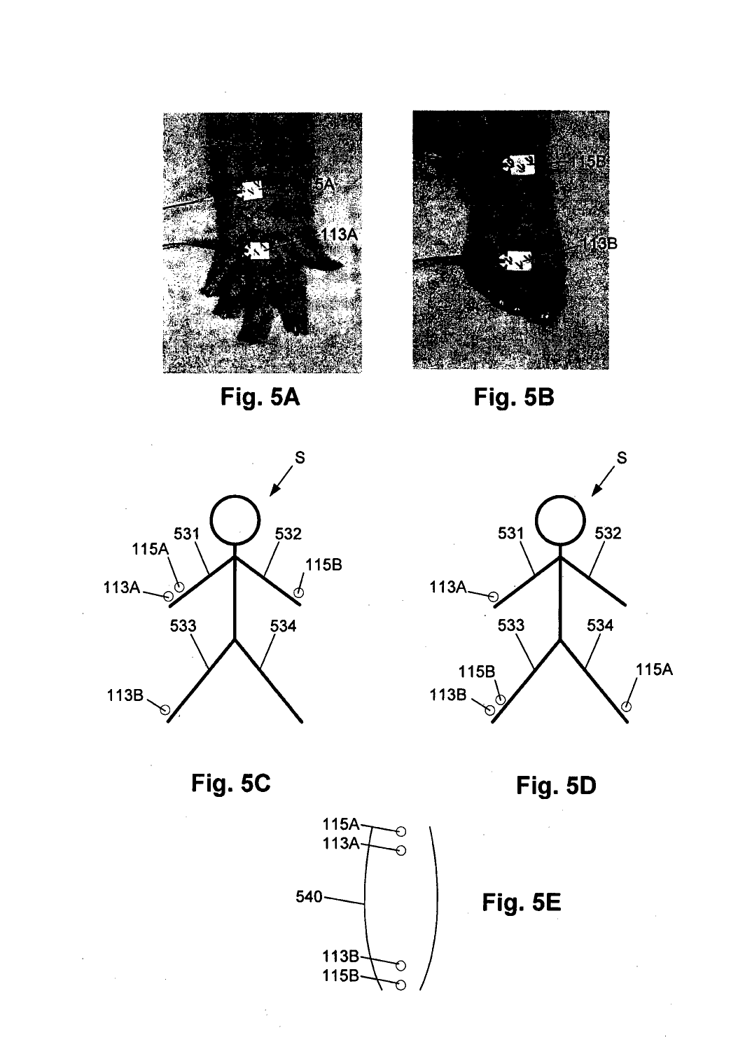

At step 420 an operator positions the electrodes on the subject S, and connects the leads 123, 124, 125, 126, to allow the impedance measurements to be performed. The general arrangement is to provide electrodes on the hand at the base of the knuckles and between the bony protuberances of the wrist, as shown in FIG. 5A, and on the feet at the base of the toes and at the front of the ankle, as shown in FIG. 5B. The configurations shown in FIGS. 5C and 5D allow the right arm 531 and the right leg 533 to be measured respectively, and it will be appreciated that equivalent arrangements can be used to measure the impedance of the left leg and left arm.

It will be appreciated that this configuration uses the theory of equal potentials, allowing the electrode positions to provide reproducible results for impedance measurements. For example when current is injected between electrodes 113A and 113B in FIG. 5C, the electrode 115B could be placed anywhere along the left arm 532, since the whole arm is at an equal potential. This is advantageous as it greatly reduces the variations in measurements caused by poor placement of the electrodes by the operator. It also greatly reduces the number of electrodes required to perform segmental body measurements, as well as allowing the limited connections shown to be used to measure each limb separately. However, it will be appreciated that any suitable electrode and lead arrangement may be used. In this regard, any suitable segment of the subject can be measured, such as any large muscle group, including but not limited to the subject's back, calf, thigh, or the like. For example, the electrode arrangement shown in FIG. 5E can be used to measure the lean tissue mass in a subject's calf 540.

At step 430 the impedance of the at risk body segments are measured. This is achieved by applying one or more current signals to the subject and then measuring the corresponding voltages induced across the subject S. It will be appreciated that in practice the signal generators 117A, 117B, and the sensors 118A, 118B, return signals to the processing system 102 indicative of the applied current and the measured voltage, allowing impedances to be determined.

Following at step 440 tissue mass and optionally fluid level impedance parameter values for each of the at risk body segments can be determined as described above. The tissue mass and fluid level impedance parameter values are typically indicative of the intracellular and extracellular resistances, and are therefore determined using impedance measurements made at one or more frequencies.

At step 450 any required reference values are selected, such as values of the constants or scaling factors, normal expected ranges, or the like. The reference is typically derived from equivalent measurements made on a reference population that is relevant to the subject under study. Thus, the population is typically selected taking into account factors such as medical interventions performed, ethnicity, sex, height, weight, limb dominance, the affected limb, or the like. Therefore if the test subject is female, with the at risk body segment being a dominant leg, then the reference values are drawn from a reference population database for the dominant leg of female subjects.

Accordingly, at this stage the processing system 102 typically accesses reference populations stored in the database, or the like. This may be performed automatically by the processing system 102 using the subject details. Thus, for example, the database may include a look-up table that specifies the normal population that should be used given a particular set of subject details. Alternatively selection may be achieved in accordance with predetermined rules that can be derived using heuristic algorithms based on selections made by medically qualified operators during previous procedures. Alternatively, this may be achieved under control of the operator, depending on the preferred implementation.

It will be appreciated by persons skilled in the art that operators may have their own reference stored locally. However, in the event that suitable references are not available, the processing system 102 can be used to retrieve a reference from a central repository, for example via an appropriate server arrangement. In one example, this may be performed on a pay per use basis.

Alternatively, in the event that a suitable reference is not available predetermined standard reference values may be used. However it will be appreciated that different values can be used as appropriate and that these values are for illustration only.

As a further alternative, the reference value may be a baseline value previously measured for the test subject. For example, in the event that a subject has suffered a debilitating injury, such as paralysis, the at risk body segment can be measured shortly after the injury and before a major loss in lean tissue mass has occurred. Changes in the measured values can then be used to accurately track changes in lean tissue mass over time.

Following this a tissue mass indicator can be determined at step 460, for example using to equation (5) or (6) above. As described above, this is typically achieved by scaling the intracellular impedance value so that the resulting indicator represents a lean tissue mass, or alternatively comparing the intracellular impedance to a baseline, so that the tissue mass indicator represents a relative change in lean tissue mass. A fluid level indicator can also optionally be determined, for example using the ratio determined from equation (8) above.

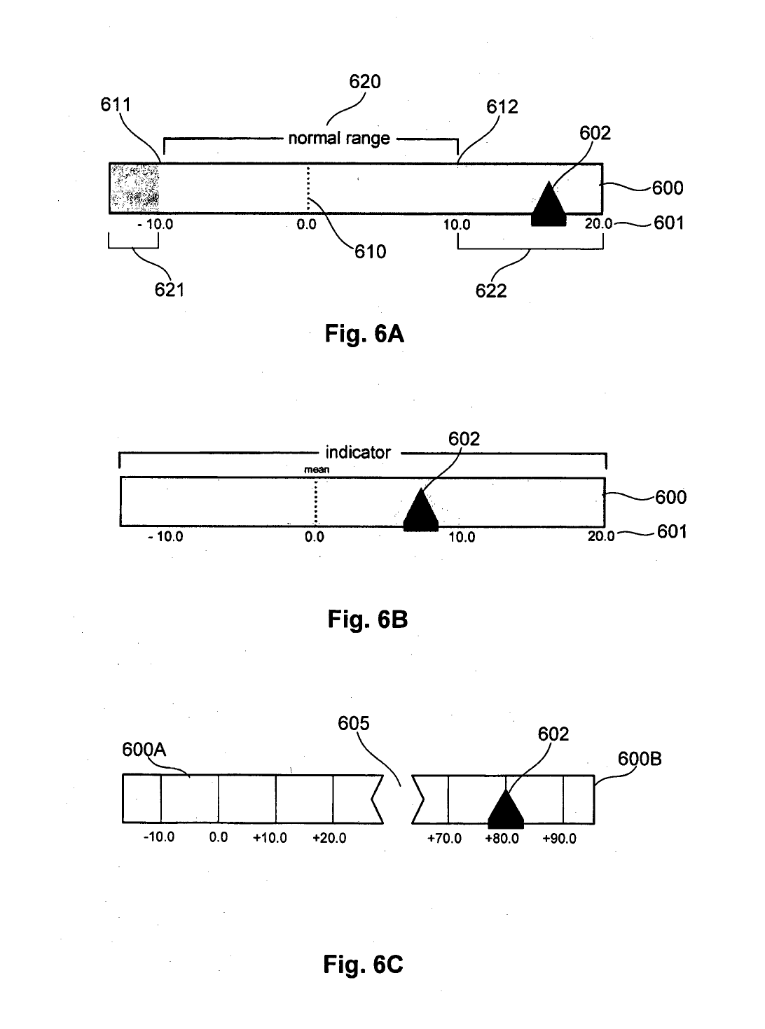

Representations of the tissue mass indicator and optionally, fluid level indicator, can then be displayed at step 470, if required, thereby allowing a healthcare professional, or other suitable individual to make an assessment of the subject's lean tissue mass. This can be achieved using graphs similar to those shown above in FIGS. 3C and 3D. However, alternative representations for the indicators can be used, as will now be described with reference to FIGS. 6A and 6B.

In these examples, the representation is in the form of a linear indicator 600, having an associated scale 601 and a pointer 602. The position of the pointer 602 relative to the scale 601 is indicative of the indicator value.

In the example of FIG. 6A, the indicator representation also includes a baseline indicator 610 representing the baseline reading for the subject, which is set to a value of “0” on the scale 601. The upper and lower thresholds are set to be a predetermined range from the baseline indicator representing a clinically relevant lean tissue mass change. This may therefore represent a change in lean tissue mass which warrants further intervention. In this example, the range thresholds are positioned at “−10” and “+10” on the scale 601 respectively, although this is not essential.

In use the lower and upper thresholds 611, 612 define a normal range 620, an investigation range 621, and an intervention range 622. The ranges can be indicated through the use of background colours on the linear indicator, so that for example, the normal range 620 is shaded green, whilst the intervention ranges 621, can be unshaded or shaded red. This allows an operator to rapidly evaluate the positioning of the pointer 602 within the ranges, allowing for fast and accurate diagnosis of medically relevant tissue mass loss. However, this is not essential, and alternatively, an absolute value of tissue mass may be displayed, depending on the preferred implementation.

In this example, the linear indicator extends up to a value of “20” as this is able to accommodate the determined value of 16.6. However, it will be appreciated that the linear indicator can be extended to any value required to accommodate the determined indicator value. To ensure that the linear scale remains clear, particularly if an extreme indicator value is to be displayed, the linear indicator 600 may include discontinuities, allowing the scale to be extended to higher values. An example of this is shown in FIG. 6C, in which a discontinuity 605 is used to separate the linear indicator 600 into two portions 600A, 600B. In this example, the linear indicator portion 600A extends from “−10” to “+20”, whilst the second linear indicator portion 600B extends from “+70” to “+90”, thereby allowing an indicator value of “80” is to be displayed by appropriate positioning of the pointer 602 in the indicator portion 605B.

Whilst a linear indicator 600 is preferred as this easily demonstrates to the operator the potential degree of severity of any tissue mass loss, this is not essential, and alternatively the scale may be modified, particularly if an outlier indicator value is determined. Thus, for example, the linear indicator could include logarithmic scaling, or the like, over all or part of its length, to allow the determined indicator value to be displayed.

In the example of FIG. 6B, no reference is available, and accordingly, the representation does not include a mean 610 or lower or upper thresholds 611, 612. In this instance, the indicator value may be an absolute value calculated using equation (5) using reference values from a reference population. To take this into account, the thresholds 611, 612, and hence the specific ranges 620, 621, 622, are excluded from the representation, highlighting to the operator that the scaled subject parameter value is indicative but not definitive of the subject's oedema status.

### EXPERIMENTAL EXAMPLES

An experimental was performed on 36 subjects by using SCI, DXA (GE Lunar, iDXA) to obtain total body (TB), right leg (RL) and left leg (LL) LTM. Additionally, measured impedance values were used to derive Rᵢ using BIS measurements performed using an Impedimed SFB7™ measuring device, which measures impedances at 256 different frequencies, using a swept frequency approach. Measurements of BIS and DXA were obtained on the same day following a 12-hour fast, while abstaining from exercise, under conditions of normal hydration, and while supine on a non-conducting surface.

Using the law of equipotential, the leads were arranged using the standard tetrapolar arrangement to determine total body (TB) impedance. To measure the RL and LL, the sense lead was moved to the dorsal surface of the left ankle at the tibia and fibular as shown in FIG. 5D, with the electrode configuration being reversed except for the drive lead that remained on the right hand.

The characteristics of the subjects are set out in Table 1, below.

TABLE 1

| | N = 36 | Range |
|---|---|---|
| Age (yrs) | 43 ± 10 | 22-62 |
| Height (cm) | 179 ± 8 | 160-196 |
| Weight (kg) | 84.6 ± 18.60 | 51-129 |
| BMI (kg/m²) | 26.3 ± 4.8 | 17-37 |
| Duration of Injury (y) | 13 ± 13 | 1-45 |
| Para/Tetra (n) | 16/20 | — |
| Complete/Incomplete (n) | 20/16 | — |

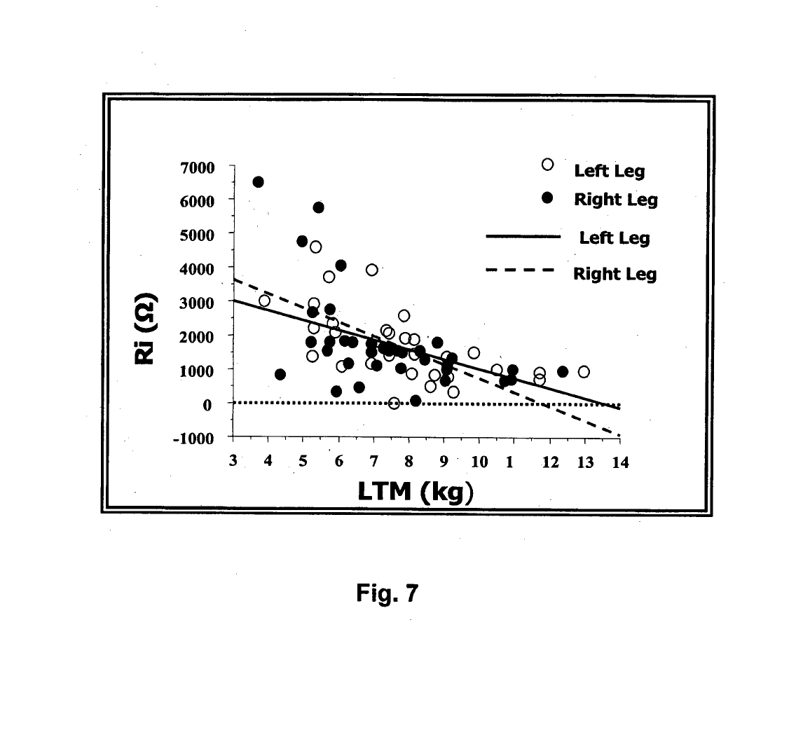

The results of a regression analysis performed on the collected data are shown in FIG. 7. These results indicate that the TB DXA L™ was inversely related to Rᵢ (r=−0.57, P<0.001). This inverse relationship was also seen between LTM and Rᵢ for the RL (r=−0.64, P<0.0001) and LL (r=−0.60, P<0.001). Duration of injury (DOI) predicted LTM and Rᵢ about equally for TB (R2=0.14, P<0.05 and R2=0.28, P<0.01; respectively), RL (R2=0.18, P<0.05 and R2=0.38, P<0.0001; respectively), and LL (R2=0.15, P<0.05 and R2=0.16, P<0.05; respectively).

The regression analysis leads to the values outlined above with respect to the scaling factor and constants, and demonstrates that changes in lean tissue mass can be tracked by monitoring changes in the impedance parameter value Rᵢ. It will be appreciated that the scaling factors and constants outlined above can be refined once additional experimental data is collated.

Persons skilled in the art will appreciate that numerous variations and modifications will become apparent. All such variations and modifications which become apparent to persons skilled in the art, should be considered to fall within the spirit and scope that the invention broadly appearing before described.

Thus, for example, it will be appreciated that features from different examples above may be used interchangeably where appropriate. Furthermore, whilst the above examples have focussed on a subject such as a human, it will be appreciated that the measuring device and techniques described above can be used with any animal, including but not limited to, primates, livestock, performance animals, such race horses, or the like.

1) A method of determining a measure of lean tissue mass for a segment of a subject, the method including, in a processing system:
determining at least one impedance value at at least one frequency, the at least one impedance value representing the impedance of the segment;
determining a tissue mass impedance parameter value using the at least one impedance value; and,
determining a tissue mass indicator based at least in part on the tissue mass impedance parameter value.

2) A method according to claim 1, wherein the tissue mass impedance parameter value is indicative of an intracellular resistance.

3) A method according to claim 1, wherein the method includes:
determining a fluid level impedance parameter value using at least one impedance value; and,
determining a fluid level indicator using at least one fluid level impedance value.

4) A method according to claim 3, wherein the at least one fluid level impedance parameter value is indicative of intracellular and extracellular fluid levels.

5) A method according to claim 4, wherein the fluid level indicator is determined based on a ratio of intracellular to extracellular resistance.

6) A method according to claim 1, wherein the method includes determining an intracellular resistance using the formula:
$${R}_{i} = \frac{{R}_{∞} {R}_{0}}{{R}_{0} - {R}_{∞}}$$

7) A method according to claim 1, wherein the method includes:
determining a first impedance parameter value from at least one impedance value measured at a first time;
determining a second impedance parameter value from at least one impedance value measured at a second time;
in the processing system, determining at least one of the tissue mass indicator and a fluid level indicator using the first and second impedance parameter values.

8) A method according to claim 7, wherein the method includes, in the processing system, determining the tissue mass indicator using the equation:
Ind=sf(Rᵢ₂−Rᵢ₁)
wherein:
Ind is the tissue mass indicator
sf is a scaling factor
Rᵢ₁ is a first impedance parameter value
Rᵢ₂ is a second impedance parameter value

9) A method according to claim 8, wherein the method includes, in the processing system, determining the tissue mass indicator using the equation:
Ind=LTM=C−sfRᵢ
wherein:
Ind is the tissue mass indicator
C is a constant
sf is a scaling factor

10) A method according to claim 9, wherein the constant C has a value between 10 and 15, and the scaling factor sf has a value of between 0.002 and 0.004.

11) A method according to claim 10, wherein the constant C has a value of 13.3, and the scaling factor has a value of 0.0033.

12) A method according to claim 8, wherein at least one of a constant and a scaling factor are determined from a reference population selected based on at least one of:
body segment dominance;
differences in body segment types;
ethnicity;
age;
gender;
weight; and,
height.

13) A method according to claim 1, wherein the method includes determining at least two impedance values including:
a first impedance value at a frequency of below 50 Hz; and,
a second impedance value at a high frequency of above 100 Hz.

14) A method according to claim 13, wherein the first impedance value is indicative of the parameter value R₀ and wherein the second impedance value is indicative of the parameter value R_{∞}.

15) A method according to claim 1, wherein the method includes:
determining a plurality of impedance values at respective frequencies; and,
using the plurality of impedance values to determine at least one of the tissue mass impedance parameter value and a fluid level impedance parameter value.

16) A method according to claim 15, wherein the method includes determining impedance parameter values by at least one of:
estimating values based on impedance measurements performed at selected respective frequencies;
solving simultaneous equations using the plurality of impedance values;
extrapolation from a plot of resistance against reactance for the plurality of impedance values;
performing a function fitting technique.

17) A method according to claim 1, wherein the method includes displaying a representation of at least one of the tissue mass indicator and a fluid level indicator.

18) A method according to claim 1, wherein the method includes in the processing system, causing one or more impedance measurements to be performed.

19) A method according to claim 1, wherein the method includes, in the processing system:
causing at least one drive signal to be applied to the subject;
determining at least one signal measured across the subject; and,
determining at least one impedance value using an indication of the drive signal and the signal measured across the subject.

20) A method according to claim 1, wherein the method includes, in the processing system:
controlling a signal generator to thereby cause the at least one drive signal to be applied to the subject; and,
determining the at least one signal measured across the subject using a sensor.

21) A method according to claim 1, wherein the method includes:
determining a number of impedance measurements, the number of impedance measurements including at least one impedance measurement at each of a number of measurement frequencies; and,
determining the impedance parameter value using the number of impedance measurements.

22) Apparatus for use in analysing impedance measurements performed on a subject, the apparatus including a processing system for:
determining at least one impedance value at at least one frequency, the at least one impedance value representing the impedance of a segment of the subject;
determining a tissue mass impedance parameter value using the at least one impedance value; and,
determining a tissue mass indicator based at least in part on the tissue mass impedance parameter value.

23) Apparatus according to claim 22, wherein the apparatus includes:
a signal generator for applying one or more electrical signals to the subject using a first set of electrodes;
a sensor for measuring electrical signals across a second set of electrodes applied to the subject; and,
a controller for:
controlling the signal generator; and,
determining the indication of the measured electrical signals.

24) Apparatus according to claim 23, wherein the controller includes the processing system.

25) Apparatus according to claim 23, wherein the processing system includes the controller.

26) (canceled)
